(19) United States
(12) Patent Application Publication
(10) Pub. No.: US 2023/0414816 A1
(43) Pub. Date: Dec. 28, 2023

(54) CAPSULE AND SYSTEM FOR DISPENSING ESSENTIAL OILS

(71) Applicant: Tammy Krutchik, Cooper City, FL (US)

(72) Inventors: Jonathan Krutchik, Ft. Lauderdale, FL (US); Tammy Krutchik, Cooper City, FL (US); Yosef Martin, Ft. Lauderdale, FL (US)

(73) Assignee: Tammy Krutchik, Cooper City, FL (US)

(21) Appl. No.: 18/214,390

(22) Filed: Jun. 26, 2023

Related U.S. Application Data
(60) Provisional application No. 63/355,721, filed on Jun. 27, 2022.

(51) Int. Cl. A61L 9/03

(52) U.S. Cl. CPC A61L 9/03; A61L 2209/133; A61L 2209/132

(57) ABSTRACT

A capsule for diffusing essential oil is disclosed. A capsule for diffusing an essential oil that has a film defining an enclosed cavity. A volume of the essential oil is within the enclosed cavity. The film of the capsule disintegrates when inserted into a diffuser and the diffuser is operating.

### CROSS-REFERENCE TO RELATED APPLICATIONS

This application is a non-provisional patent application which claims the benefit of the filing date of U.S. Provisional Application Ser. No. 63/355,721 titled “CAPSULE AND SYSTEM FOR DISPENSING ESSENTIAL OILS” and filed Jun. 27, 2022, and the subject matter of which is incorporated herein by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of aromatherapy, and more specifically to the field of essential oil and essential oil diffusers.

### BACKGROUND

Essential oils are extracts derived from plants and they often have attractive aromas and health benefits. Historians believe that the practice of keeping and collecting essential oils began in ancient India, and their continued use and spread across the globe is a testament to their longevity and popularity. Essential oils have been used in many ways, the most popular of them being to scent home and utilize their aromatherapeutic properties to act as a de-stressor. Essential oils are so effective that doctors recommend using them to boost mood, reduce stress, and improve sleep. 11 Essential Oils: Their Benefits and How To Use Them, CLEVELAND CLINIC (Dec. 14, 2021) https://health.clevelandclinic.org/essential-oils-101-do-they-work-how-do-you-use-them/. With the growth of the Internet and the increased popularity of essential oils on social media, people have gotten more informed about the potential benefits of essential oils and tried to bring essential oils into their homes to utilize these benefits.

Despite their beneficial potential, essential oils can have some effects if used improperly. Essential oils can have powerful aromas which, if used too strongly, could cause headaches or irritations to some people. Additionally, some aromas could clash together and cause a bad smell rather than a pleasant one. Essential oils are growing increasingly popular, which is increasing their availability and the number of varieties that are accessible to the public. However, with this increase in availability, consumers are being asked to memorize a growing number of species, varieties, and individual benefits attributed to each essential oil. There is a steep barrier to entry that can intimidate those who want to begin to learn about essential oils, which can be preventative and keep consumers from integrating the potential benefits that essential oils could have on their lives.

Additionally, the distribution of essential oils can pose an environmental toll through its packaging. Essential oils are often distributed in opaque plastic or glass bottles and containers which are intended to be disposed of after use. These bottles are unideal as they take up lots of room to store, utilize a dropper dispensing system that often creates wasted oil and a mess, and end up in landfills after they are emptied. The glass bottles can be difficult to recycle if not properly cleaned and plastic bottles and packaging are often completely un-recyclable. These factors together mean that the packaging for essential oils is contributing to the growth of landfills, which is of great concern to customers who want to use essential oils but also want to be environmentally conscious and limit the amount of waste they produce.

As a result, there exists a need for improvements over the prior art and more particularly for a more efficient way of allowing consumers a cost-effective, eco-friendly, and convenient way to diffuse essential oils within their home without having to learn about the hundreds of varieties of essential oils.

### SUMMARY

A capsule and system for diffusing essential oils is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a capsule for diffusing essential oil is disclosed. A capsule for diffusing an essential oil or any other type of liquid, including liquid with fragrances. The capsule has a film defining an enclosed cavity. A volume of the essential oil is within the enclosed cavity. The film of the capsule disintegrates when inserted into a diffuser and the diffuser is operating. The film comprises at least one of a water-soluble material, a material that breaks when a sound wave at a certain frequency is applied to the material, and a material that breaks when a certain amount heat is applied to the material.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a perspective view of a capsule that shows the opaque film of the capsule and the enclosed cavity which contains a volume of essential oil or other type of liquid, according to a sample embodiment;

FIG. 2 is a side view of a diffuser that contains an opening, an area to receive the capsule and hold a conductor such as water, a heating unit, and a power source, according to a sample embodiment;

FIG. 3 is a diagram of a chemical structure of a poly vinyl alcohol molecule, according to a sample embodiment; and

FIG. 4 is a diagram of a chemical structure of a poly methyl acrylate molecule, according to a sample embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing a system that allows the consumer the ability to quickly and easily diffuse the correct volume of essential oils with the diffuser in a safe and eco-friendly way. The opaque capsule allows the consumer to always disperse the right volume of essential oils and avoids overuse or unpleasant mixing of oils or other liquid. Other inventions in the past have facilitated the diffusion of oils or fragrance, however, they have required the consumer to understand the species and intricate details about the oils in order to utilize their potential benefits. Past inventions also do not pre-portion out the volume of required oils which means that the consumer risks putting too much oil or other liquid into the diffuser which can cause headaches through a too pungent smell, or too little of the oil, which does not fully take advantage of the potential benefits from the essential oils. This invention also results in a spill-proof method of introducing oil to the diffuser. The capsule is clean, not sticky, and easy for the user to handle and store. Current dispensers utilize a system that requires a numerous number of bottles that dispense on a per drop basis. These bottles often utilize a dropper built into the cap which tends to run down the side of the bottle when being withdrawn. This is unideal as it creates wasted oil and a mess for the user.

Additionally, the slow disintegration of the capsule allows the capsule to release over time without intervention from the user, which lets the user enjoy the scent without being forced to get up and constantly insert new oils or other liquid. The previous prior art has required constant maintenance from the user, whereas this system facilitates a steady, long-term supply of essential oil particles. This system also can allow for a variation within the release of the oils or other types of liquids through control over the thickness of the film. The film breaks down when placed into the diffuser while the diffuser is operating. Operating may be defined as having the capsule within the diffuser while the diffuser contains water or another type of liquid conductor. In some embodiments, the rate of the breakdown, dissolving or disintegration, and subsequent release of the oils, may be varied through the thickness of the film as a thicker film would take longer to dissolve and a thinner film could be dissolved rather quickly. Therefore, the film could facilitate a timed release of oils or fragrance by having varying thicknesses of films that dissolve at different rates and delay the release of certain oils or other types of liquids over a period of time. However, other means of causing a time release may be used and is within the spirit and scope of the present invention. Furthermore, the opaque capsule and dispersal system provides an environmentally friendly way to utilize oils or other types of liquids and eliminates the need for excess packaging waste. With the proposed system, consumers can receive the needed volume of oil without excess packaging waste or the need to collect dozens of small bottles of essential oils, which occupies unnecessary room in the household.

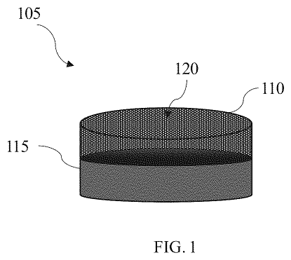

Referring now to the Figures, FIG. 1 is a perspective view of a capsule 105 that comprises: a film defining an enclosed cavity; a volume of the essential oil or fragrance within the enclosed cavity; and wherein the film disintegrates when inserted into a diffuser and the diffuser is operating. As mentioned above, operating may be defined as having the capsule within the diffuser while the diffuser contains water or another type of liquid conductor. A capsule is defined as a small case or container that is made to contain another substance such as another liquid, solid, or powder. Other common forms of capsules include pills and laundry detergent “pods”. The film 110 comprises at least one of: a water-soluble material, a material that breaks when a sound wave at a certain frequency is applied to the material, and a material that breaks when a certain amount of heat is applied to the material. Additionally, the film comprises at least one of a poly vinyl alcohol, a co-polymer of poly vinyl alcohol, and poly methyl acrylate. The film could also be comprised of other chemical compositions and these compositions may be used and are within the scope and spirit of the present invention. The film could have light-protective properties, such as an opaque tint, to prevent damage to the essential oil's chemical structure due to light exposure and to protect the potency of the oils.

The film defines a volume of the essential oil or fragrance disposed within the enclosed cavity 120. The capsule has a volume of oil or fragrance 115 inside it and, generally, the volume is approximately 0.25 ml. The specifications of the volume are determined by the type of oil or fragrance and the desired potency of the oil or fragrance. Other volumes, either less than or more than 0.25 ml could also be used and should be considered with the scope and the spirit of the invention. FIG. 1 shows one volume of oil or fragrance; however, the capsule could also contain multiple types of oil or fragrance that mix together in the enclosed cavity. Additionally, the enclosed cavity could contain multiple pockets created by the film to separate different types of oil or fragrance. In that embodiment, for example, the oils or other types of liquids would be separated internally through walls of film and would mix together as the capsule broke down. Any number of pockets, whether singular or plural, are within the spirit of the invention and should be considered within the scope of the claims. Furthermore, the film within this system also can allow for a variation within the release of the oils or other types of liquids through control over the thickness of the film. The film breaks down when placed into the diffuser while the diffuser is operating. The rate of the break down, and subsequent release of the oils, could be varied through the thickness of the film as thicker film would take longer to dissolve and thinner film could be dissolved rather quickly. Therefore, the film could facilitate a timed release of oils or fragrance by having varying thicknesses of film which dissolves at different rates and delays the release of certain oils or other types of liquids. In the present embodiment, the capsule is shown in the shape of a cylinder. However, the invention is not limited to that shape. Other embodiments may be modeled into the shape of a cube or a square, however other shapes such as circular or triangular may also be used and are within the spirit and scope of the present invention.

The capsule 105 comprises at least one of: a water-soluble material; a material that breaks down when a sound wave at a certain frequency is applied to the material; and a material that breaks when a certain amount of heat is applied to the material. All the materials are designed to hold together not only throughout the packaging, shipping, and delivery process to the consumer but also for the film to dissolve when inserted into a diffuser and the diffuser is operating. Additionally, the film is designed to protect the chemical integrity of the essential oils or fragrance inside of it, so that the consumer receives a potent oil or fragrance that will disperse the most effective amount of oil or fragrance into the consumer's household. The capsule is made of certain properties so that the capsule dissolves or disintegrates over a period of time when the capsule is received by the diffuser and the diffuser is operating. Operating may be defined as having the capsule within the diffuser while the diffuser contains water or another type of liquid conductor. As the capsule disintegrates or dissolves, the particle of the essential oil or fragrance will be diffused into the air.

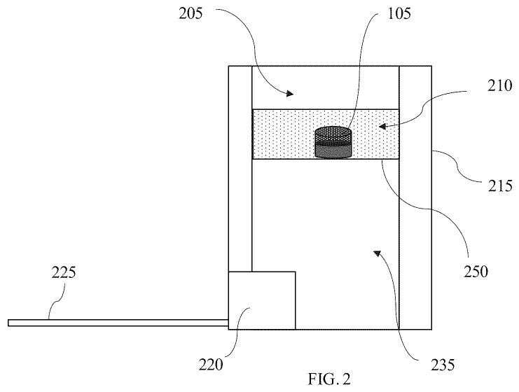

FIG. 2. is a side view of a diffuser that contains an opening, an area to receive the capsule and hold a conductor such as water, a heating unit or heater 220, and a power source. FIG. 2 shows a cross-section of a diffuser. A diffuser is a device that breaks the molecules of a substance, such as an essential oil or fragrance, into smaller molecules so that they may be spread into the air. There are several types of diffusers and the most common is ultrasonic, which uses sound waves to create a mist. Other embodiments use heat or a fan to create evaporation and disperse oil and fragrance molecules into the air. All diffusers tend to spread the molecules into the air and help the molecules to disperse into all parts of a room. Diffusers may be a device with the single purpose of diffusing molecules, but they may also be paired with other uses such as being a light source. A diffuser model with more than one purpose is within the spirit of this invention and should be considered within its scope. All the following elements are housed within a body 215, which provides a secure covering for the internal components and creates inner walls for the receiving area. The body 215 is configured to rest on the floor and is configured to hold the internal workings of the invention such as the receiving area 205, the empty space 235, the bottom edge 250 of the receiving area, the capsule 105 (if inserted by the customer), and the heater 220 up and perpendicular to the ground.

The body may include material such as carbon steel, stainless steel, aluminum, Titanium, other metals or alloys, composites, ceramics, polymeric materials such as polycarbonates, such as Acrylonitrile butadiene styrene (ABS plastic), Lexan™, and Makrolon™. However, other types of materials may also be used and are within the spirit and scope of the present invention. The body may be formed from a single piece or from several individual pieces joined or coupled together. The components of the body may be manufactured from a variety of different processes including an extrusion process, a mold, welding, shearing, punching welding, folding etc. However, other types of processes may also be used and are within the spirit and scope of the present invention. The body may be modelled into the shape of a cube or a square, however other shapes such as circular or triangular may also be used and are within the spirit and scope of the present invention. Additionally, the present embodiment shows the receiving area to be the same square shape as the body. However, the body may be threaded and in different shapes. For example, the body may be in the shape of a square and the receiving area could be in the shape of a circle. All combinations of shapes may also be used and are within the spirit and scope of the present invention.

In the present embodiment, the body 215 is shown to be in a vertical position so that it is taller than it is wider. However, the body of the diffuser could also be in a horizontal position in which the body is wider than it is taller. Additionally, the diffuser could also be in any unconventional shape, and it still may be used and should be considered to be within the scope and spirit of the invention. For example, the diffuser could be modeled into the shape of a fish and this unconventional shape would still contain all the inventive aspects and may be used as it should still be considered within the spirit and the scope of the invention.

The receiving area 205 is open-ended so that a capsule 105 may be received by the diffuser. In the present embodiment the receiving area is shown at the top of the diffuser so that the receiving area opens towards the open air. The receiving area creates a well within the diffuser in which the capsule may rest. However, in other embodiments, the receiving area could be put within any part of the body 215. For example, instead of an open air well, another embodiment could have the receiving area to be in the form of a window. In that embodiment, the body would have an opening that is cut out in one of the body walls through which the consumer inserts the capsule. In both embodiments, the capsule would sit inside of the receiving area to disintegrate and dissolve due to the heater 220. The receiving area may contain a liquid conductor 210 such as water or another type of oil that reacts with the material that makes up the film of the capsule. The receiving area holds a liquid conductor which may be heated or vibrated to break the capsule apart over time while the diffuser is operating. However, the receiving area may also not interact with the heater 220 and passively break down the capsule through the interactions between the film of the capsule and the liquid conductor. The bottom edge 250 of the receiving area is a closed end and provides a base for which capsule and the liquid conductor. Below the bottom edge 250 of the receiving area, is an empty space 235. In the present embodiment, the space is shown as empty, however, it could be used to house liquid conductors such as water or oil.

Additionally, this space houses a heater 220 which is used to heat the conductors and break apart the capsule 105. Heat may be used to help break apart the film around the capsule. In the present embodiment, a heater is shown, however, a sound-producing unit that creates sound waves that break down the film of the capsule, could also be used. Furthermore, the heater could also have an additional function such as producing light. In that embodiment, a heater such as a heat-producing bulb could be used to both create heat and produce light so that the diffuser acts as both a light source and an aroma diffuser. Multi-functionality and additional uses of the empty space and the heater are within the scope and spirit of the invention. The heater 220 is powered by a power source 225 which could be plugged into a wall outlet or a battery. The empty space 235 could be used to house a battery or an additional power source if the connection to a wall outlet is not desired.

Additionally, the present embodiment shows the catalyst of the disintegration or dissolution of the capsule to be initiated by heat, however other catalysts may be used. For example, in another embodiment, the capsule could be dissolved solely through long-term exposure to water or a slightly corrosive type of oil or chemical. In that embodiment, heat may be used to speed up the process rather than to initiate the process. In a different embodiment, the capsule could be broken apart when a sound wave at a certain frequency is applied to the material that makes up the film. In that embodiment, the sound wave would be at either a very low pitch or a very high pitch so that it would not be heard by the consumer, but it still would have enough energy in the frequency to disrupt the film's molecules and break it apart so that the essential oil's particles are still released into the household's air. Similarly, to the other embodiments, the heater could still be utilized to help the diffusion process and ensure that the essential oil particles are dispersed in a slow and consistent manner.

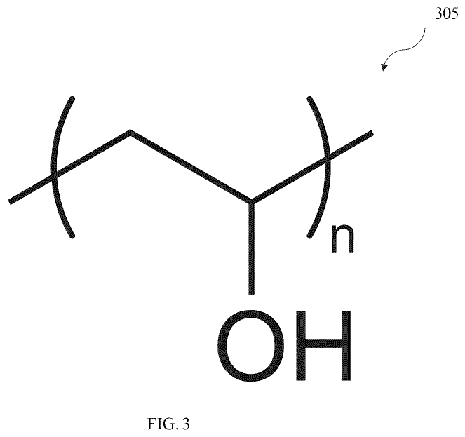

FIG. 3 is a diagram of a chemical structure of a poly vinyl alcohol molecule, according to a sample embodiment. Poly vinyl alcohol (“PVA”) 305 is a water-soluble synthetic polymer. A polymer is a natural or synthetic substance that is made of macromolecules and is an extremely common component of the natural world. Some of the most known polymers are cellulose, which makes up most of a plant's structure, and fibrous materials like wool, silk, and hemp. Synthetic polymers are also extremely common and have been used in development and manufacturing for years. Polymers make up plastics, including some of the most well-known synthetic materials such as nylon, neoprene, polyethylene, and silicone. PVA is a flexible, colorless, and odorless polymer that has been used in many applications such as for vascular stents and contact lenses. PVA is a well-known and safe material that will break down when exposed to heat and water which facilitates the slow disintegration of the capsule and the steady release of essential oils or other types of liquids.

In the present embodiment, the capsule film could be formed with PVA to create an enclosed cavity 120 which can be filled with a volume of essential oils. PVA can also be colored and dyed so that the film of the capsule can be opaque and therefore protective of the light-sensitive essential oils inside of it. Additionally, the enclosed cavity could contain multiple pockets created by the film to separate different types of oil or fragrances. In that embodiment, for example, the oils would be separated internally through walls of film and would mix together as the capsule broke down. Any number of pockets, whether singular or plural, are within the spirit of the invention and should be considered within the scope of the claims.

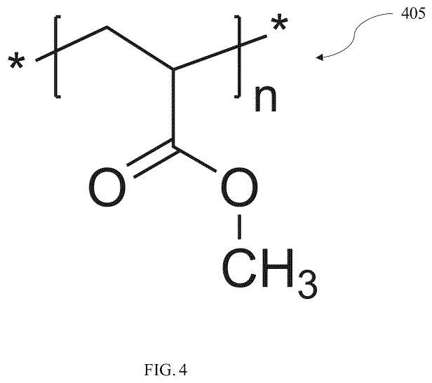

FIG. 4 is a diagram of a chemical structure of a poly methyl acrylate molecule, according to a sample embodiment. Poly methyl acrylate (“PMA”) 405 is a hydrophobic synthetic polymer. A polymer is a natural or synthetic substance that is made of macromolecules and is an extremely common component of the natural world. Some of the most known polymers are cellulose, which makes up most of a plant's structure, and fibrous materials like wool, silk, and hemp. Synthetic polymers are also extremely common and have been used in development and manufacturing for years. Polymers make up plastics, including some of the most well-known synthetic materials such as nylon, neoprene, polyethylene, and silicone. PMA will break down when exposed to heat and water which facilitates the slow disintegration of the capsule and the steady release of essential oils or other types of liquids.

In the present embodiment, the capsule film could be formed with PMA to create an enclosed cavity 120 which can be filled with a volume of essential oils. PMA can also be colored and dyed so that the film of the capsule can be opaque and therefore protective of the light-sensitive essential oils inside of it. Additionally, the enclosed cavity could contain multiple pockets created by the film to separate different types of oil or fragrances. In that embodiment, for example, the oils or other types of liquids would be separated internally through walls of film and would mix together as the capsule broke down. Any number of pockets, whether singular or plural, are within the spirit of the invention and should be considered within the scope of the claims.

Additionally, the film of the capsule could be composed of a co-polymer of poly vinyl alcohol and poly methyl acrylate (PVA-PMA). A copolymer is a combination polymer that is made from two different species of monomers. In that embodiment, the polymers of PVA and PMA would be combined to utilize the beneficial properties of both polymers and to form a biopolymer. The copolymer potential, however, is not limited to a combination of only two polymers, and different embodiments could combine several polymers into terpolymers, quarter polymers, and all other forms of polymers. Other combinations of polymers, chemicals, and synthetic or natural materials could be utilized and should be considered within the spirit and scope of the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A capsule for diffusing an essential oil, wherein the capsule comprises:
a film defining an enclosed cavity; and
a volume of the essential oil within the enclosed cavity;
wherein the film disintegrates when inserted into a diffuser and the diffuser is operating.

2. The capsule of claim 1, wherein the film comprises at least one of:
a water soluble material;
a material that breaks when a sound wave at a certain frequency is applied to the material; and
a material that breaks when a certain amount heat is applied to the material.

3. A capsule for diffusing an essential oil comprising:
a water soluble film defining an enclosed cavity; and
a volume of the essential oil disposed within the enclosed cavity;
wherein the water-soluble film dissolves when inserted into a diffuser and the diffuser is operating.

4. The capsule of claim 2, wherein the water-soluble material comprises at least one of a poly vinyl alcohol (PVA), a co-polymer of poly vinyl alcohol and poly methyl acrylate (PVA-PMA).

5. A system for dispensing an essential oil, wherein the system comprises:
a diffuser for diffusing the essential oil into the air; and
a capsule configured to be received by the diffuser, wherein capsule comprises;
a film defining an enclosed cavity; and
a volume of the essential oil disposed within the enclosed cavity;
wherein the capsule is made of certain properties so that the capsule dissolves or disintegrates over a period of time when the capsule is received by the diffuser and the diffuser is operating; and
wherein when the diffuser is operating, the essential oil inside the capsule is diffused into the air by the diffuser as the capsule disintegrates.

6. The capsule of claim 1, wherein the film comprises light protective properties so that does effect the essential oil.

7. The capsule of at least one of claims 1, 3 and 5, wherein the volume is approximately 0.25 ml.
